(19) United States
(12) Patent Application Publication
(10) Pub. No.: US 2025/0249247 A1
(43) Pub. Date: Aug. 7, 2025

(54) Electrical Stimulation Vector Potential Coil Device For Use In Ophthalmic Treatment

(71) Applicants: SUMIDA CORPORATION, Tokyo (JP); NATIONAL UNIVERSITY CORPORATION IWATE UNIVERSITY, Iwate (JP)

(72) Inventors: Kenji TERAO, Miyagi (JP); Masaki SAITO, Miyagi (JP); Yosuke ARAKAKI, Miyagi (JP); Masahiro DAIBO, Morioka (JP)

(21) Appl. No.: 19/187,349

(22) Filed: Apr. 23, 2025

Related U.S. Application Data
(63) Continuation of application No. PCT/JP2023/026195, filed on Jul. 18, 2023.

(30) Foreign Application Priority Data
Dec. 16, 2022 (JP) 2022-200917

(51) Int. Cl. A61N 1/36

(52) U.S. Cl. CPC A61N 1/36046

(57) ABSTRACT

A vector potential coil is a solenoid coil extending along a coil axis which is curved. A ferromagnetic member extends in an inside of the solenoid coil along the coil axis. A power supply device conducts a current through the vector potential coil. The vector potential coil and the ferromagnetic member have openings in a circumferential direction.

### CROSS REFERENCE TO RELATED APPLICATIONS

This application is a continuation of PCT Application No. PCT/JP2023/026195, filed on Jul. 18, 2023, which claims priority to Japanese Patent Application No. 2022-200917, filed on Dec. 16, 2022. The contents of both of the above applications are expressly incorporated herein by reference in their entirety.

### BACKGROUND

### Technical Field

The present invention relates to an electrical stimulation vector potential coil device for use in an ophthalmic treatment and a method of using the same.

### Related Art

In recent years, the great progress has been made in research into treatment methods for myopia, and ophthalmologic diseases, such as ptosis, entropion, lagophthalmos, stye, chalazion, blepharospasm, eyelid malignant tumor, conjunctivitis, dermoid, subconjunctival hemorrhage, pinguecula, malignant lymphoma, scleritis, scleromalacia, keratitis, keratoconus, pterygium, granular corneal dystrophy, rodent corneal ulcer, uveitis, cataract, ectopia lentis, luxatio lentis, exfoliation syndrome, vitreous hemorrhage, asteroid hyalosis, persistent hyperplastic primary vitreous, glaucoma, ocular hypertension, retinal detachment, diabetic retinopathy, age-related macular degeneration, polypoidal choroidal vasculopathy, retinal artery occlusion, retinal vein occlusion, retinitis pigmentosa, retinoschisis, macular hole, epiretinal membrane, coats disease, familial exudative vitreoretinopathy, acute retinal necrosis, cytomegalovirus retinitis, retinopathy of prematurity, retinoblastoma, optic neuritis, optic nerve hypoplasia, Leber's hereditary optic neuropathy, optic canal fracture, multiple sclerosis, Devic's disease, pituitary tumor, cerebral infarction, dry eye, dacryocystitis, nasolacrimal duct obstruction, canaliculitis, myopia, hyperopia, astigmatism, presbyopia, strabismus, inferior oblique hypermobility, Duane syndrome, oculomotor paralysis, abducens nerve paralysis, Horner syndrome, Adie syndrome, traumatic mydriasis, color vision deficiency, myasthenia gravis, systemic lupus erythematosus, and thyroid eye disease. Among these disease treatments, the research into treatment methods and devices using an electrical stimulation has been particularly attracting attention.

For instance, in order to treat a patient with a history of age-related macular degeneration, the following has been disclosed (for instance, refer to Japanese Patent Publication Number 2005/529689): An electrical nerve stimulator is used so as to apply a direct current burst of a high frequency for a short period of time, and after that, apply a current burst of a low frequency for a long period of time. Specifically, the electrical nerve stimulator is packaged so that no user input is required, and with which a user simply applies electrodes to the correct body parts so as to initiate the current sequence being programmed in advance. In addition, in order to treat a disease such as retinal detachment, a treatment method has been disclosed (for instance, refer to Non-Patent Document 1: Internet <URL: http://www.nannbyou.com/medical/me37.html>). Specifically, in the treatment method, an acupuncture and moxibustion needle is passed through a special acupressure point at a back of an eyeball and stimulated with a weak current. An electrical signal is converted into electrical energy at photoreceptor cells in a retina, thereby restoring eyeball tissues such as the retina and a vitreous body to health.

On the other hand, a vector potential generation device has been developed (for instance, refer to International Patent Publication Number WO2015/099147). The vector potential generation device generates a vector potential by conducting a current through a vector potential coil that is formed by circulating a solenoid coil. Furthermore, a vector potential detection device has also been developed (for instance, refer to Japanese Patent Number 6950925). The vector potential detection device detects a vector potential by utilizing a state in which a voltage is induced by a temporal change (varying in time) of the vector potential.

In the above-mentioned electrical stimulation methods, it is necessary that the electrodes are attached to the skin or special acupuncture and moxibustion needles are inserted into the muscles. Therefore, there is a concern about applying an invasion (a stress to or injuring) a living body (organism) during the ophthalmic treatment or operation. Therefore, a non-invasive (non-stress and non-injuring) treatment device, while having the effect of an electrical stimulation, which can minimize damage to such as the living body, is expected.

### SUMMARY

The present invention has been made in view of the above and has an object that is to obtain an electrical stimulation vector potential coil device for use in an ophthalmic treatment, while applying an appropriate electrical and/or magnetic stimulation to a living body, and at the same time, avoiding an invasion (a stress to or injuring) to the living body.

An electrical stimulation vector potential

generation device used in an ophthalmic treatment according to the present invention includes a vector potential coil which is a solenoid coil extending along a straight (linear) coil axis or a curved coil axis, a ferromagnetic member extending along the coil axis within the solenoid coil, and a power supply device that conducts a current through the vector potential coil. The vector potential coil and the ferromagnetic member have openings in a circumferential direction.

### EFFECTS OF THE INVENTION

According to the present invention, for instance, it is possible that a vector potential coil device can be placed at a position that is away from a living body by several millimeters to ten and several centimeters and an electric field and/or a current can be applied from that position. As a result, an invasion (stress or injuring) degree to the living body can be reduced. Therefore, it is possible to obtain a vector potential coil device that performs an ophthalmic treatment or an ophthalmic operation through an electrical stimulation, while eliminating the invasion (stress or injuring) caused by electrodes or acupuncture and moxibustion needles for the electrical stimulation to the living body.

### BRIEF DESCRIPTION OF THE DRAWINGS

FIG. 1 is a block diagram that shows a configuration of a vector potential generation device 10 according to an embodiment of the present invention.

FIG. 2 is a diagram that shows an example of a vector potential coil with respect to a vector potential coil device 1 shown in FIG. 1.

FIG. 3 is a diagram that shows an example of a vector potential coil device 1 according to a first embodiment.

FIG. 4 is a diagram that shows an example of an application of a vector potential by a vector potential coil device 1 according to the first embodiment.

FIG. 5 is a diagram that shows an example of a support body 110 of the vector potential coil device 1 according to the first embodiment.

FIG. 6 is a diagram that shows an example of how to attach the vector potential coil device 1 according to the first embodiment.

FIG. 7 is a diagram that shows a configuration of a vector potential coil device 1 with respect to a vector potential generation device 10 according to a second embodiment of the present invention.

FIG. 8 is a diagram that shows a configuration of a vector potential coil device 1 with respect to a vector potential generation device 10 according to a third embodiment of the present invention.

FIG. 9 is a diagram that shows a configuration of a vector potential coil device 1 with respect to a vector potential generation device 10 according to a fourth embodiment of the present invention.

FIG. 10 is a diagram that shows a configuration of a vector potential coil device 1 with respect to a vector potential generation device 10 according to a fifth embodiment of the present invention.

FIG. 11 is a diagram that shows a configuration of a vector potential coil device 1 with respect to a vector potential generation device 10 according to a sixth embodiment of the present invention.

FIG. 12 is a diagram that shows a configuration of a vector potential coil device 1 with respect to a vector potential generation device 10 according to a seventh embodiment of the present invention.

FIG. 13 is a front view that shows an example of a vector potential coil with respect to a vector potential coil device 1 according to an eighth embodiment of the present invention.

FIG. 14 is a top view that shows an example of the vector potential coil with respect to the vector potential coil device 1 according to the eighth embodiment of the present invention.

FIG. 15 is a side view that shows an example of the vector potential coil with respect to the vector potential coil device 1 according to the eighth embodiment of the present invention.

FIG. 16 is a diagram that shows an example of a vector potential coil with respect to a vector potential coil device 1 according to a ninth embodiment of the present invention.

FIG. 17 is a diagram that shows an example of attaching the vector potential coil device 1 according to the ninth embodiment of the present invention.

FIG. 18 is a diagram that shows an example of attaching the vector potential coil device 1 according to the ninth embodiment of the present invention.

FIG. 19 is a diagram that shows an example of an application of a vector potential by a vector potential generation device 1 according to another embodiment. In

FIG. 19, the vector potential generation device 1 has a vector potential coil 11, a magnetic shield, and an electrostatic shield.

FIG. 20 is a diagram that shows an example of an application of a vector potential by a vector potential generation device 1 according to another embodiment. In FIG. 20, the vector potential generation device 1 has a vector potential coil 11 and either a magnetic shield or an electrostatic shield.

### DETAILED DESCRIPTION

Embodiments of the present invention will be explained below with reference to the drawings.

### First Embodiment

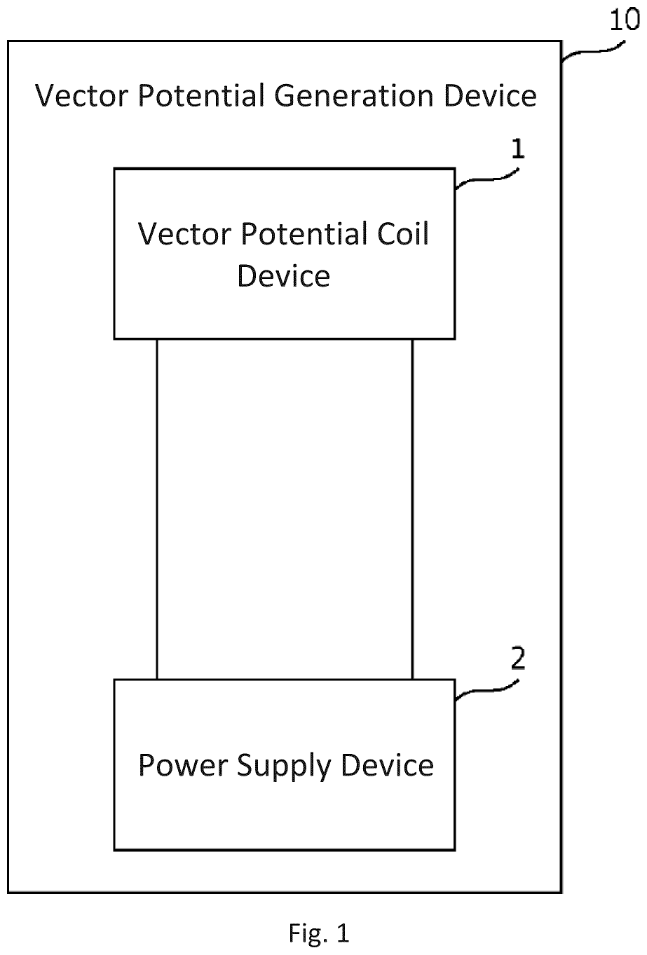

FIG. 1 is a block diagram that shows a configuration of a vector potential generation device 10 according to an embodiment of the present invention. The vector potential generation device 10 shown in FIG. 1 has a vector potential coil device 1 and a power supply device 2.

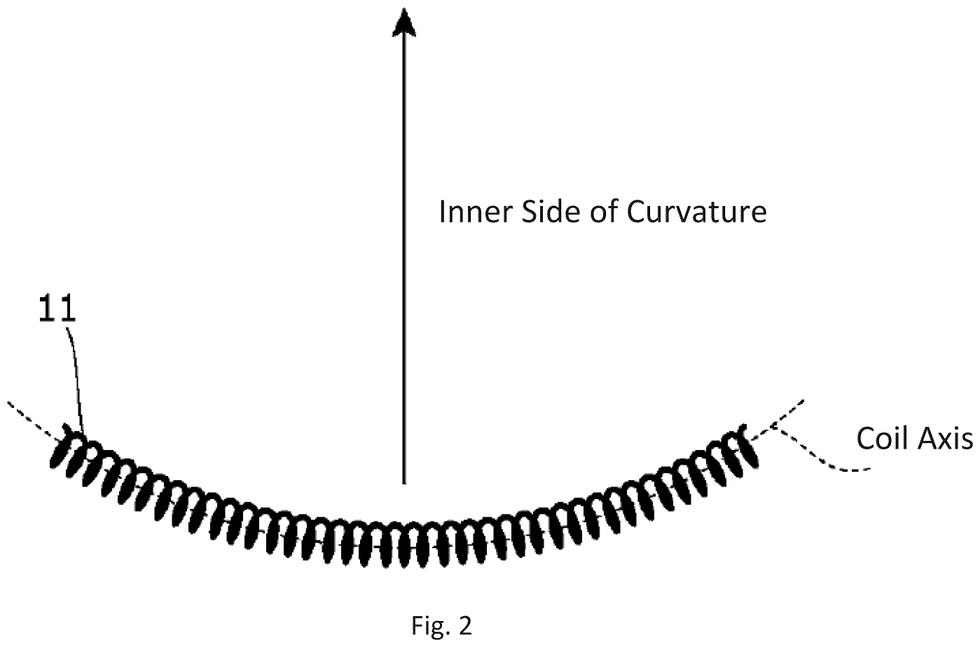

FIG. 2 is a diagram that shows an example of a vector potential coil with respect to the vector potential coil device 1 shown in FIG. 1. The vector potential coil device 1 has, as shown in, for instance, FIG. 2, the vector potential coil (also referred to as “a VP coil” below) 11. The VP coil 11 is a solenoid coil extending along a curved coil axis.

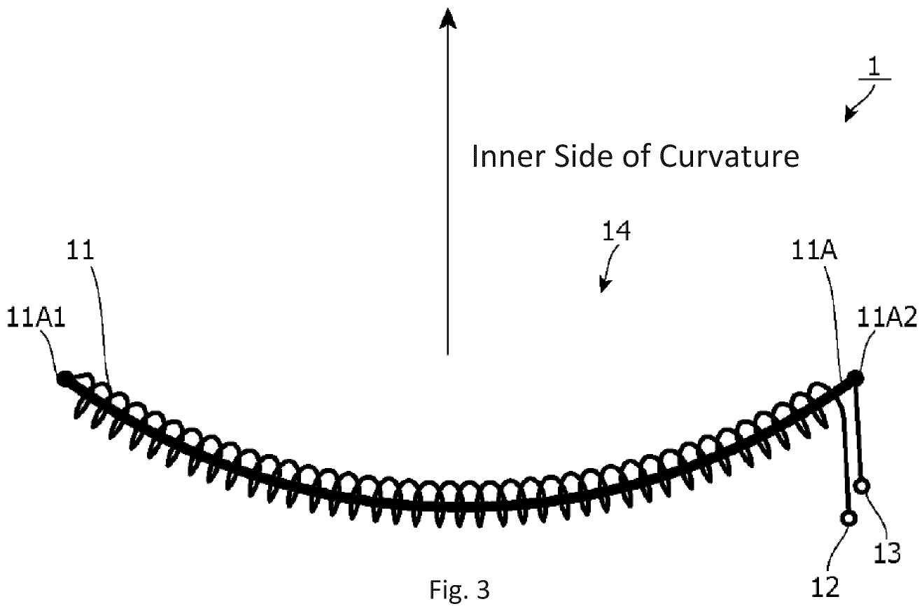

FIG. 3 is a diagram that shows an example of a vector potential coil device 1 according to a first embodiment. As shown in, for instance, FIG. 3, the vector potential coil device 1 has a ferromagnetic member 11A in addition to the above-mentioned VP coil 11. The ferromagnetic member 11A is in a shape that extends along the above-mentioned coil axis within the above-mentioned solenoid coil and is formed with a ferromagnetic material.

A vector potential due to a current that is conducted through the VP coil 11 is weakened as it moves away from the current. However, since the VP coil 11 and the ferromagnetic member 11A are curved as mentioned above, an intensity of the vector potential becomes greater at an inner side (an inner direction) of the curvature (a curvature center in the case of a circular arc shape). Specifically, the vector potentials being generated by the current at each position of the VP coil 11 overlap at the inner side of the curvature. Further, since the vector potential is enhanced according to an effective magnetic permeability of the ferromagnetic member 11A, the intensity of the vector potential becomes greater at the inner side of the curvature (a curvature center in the case of a circular arc shape).

The power supply 2 shown in FIG. 1 generates a current based on power from, for instance, a commercial power supply or a battery (a primary battery or a secondary battery), and conducts that current (here, an alternating current (AC current) of a predetermined frequency) through the VP coil 11. Note that in a case in which the power supply device 2 conducts a current through the VP coil 11 by using the power from the battery, the vector potential generation device 10 may be a portable device that incorporates the battery.

In addition, as shown in, for instance, FIG. 3, the VP coil 11 and the ferromagnetic member 11A have an opening 14 in a circumferential direction. In other words, a coil axis of the VP coil 11 does not go around once (one revolution) or more.

For instance, the above-mentioned coil axis is in a circular arc shape. Further, an angle (central angle) from one end to the other end of the VP coil 11 (the coil axis of the VP coil) when viewed from a center of a circle including the coil axis (i.e., the circular arc) is less than 360 degrees. Similarly, an angle (central angle) from one end to the other end of the ferromagnetic member 11A when viewed from the center of the circle including the coil axis is less than 360 degrees. As a result, the opening 14 is formed. For instance, the central angle may be 180 degrees or may be less than 180 degrees. However, the larger the central angle is, the greater the intensity of the vector potential in the inner side of the curvature becomes. Thus, it is preferred that the central angle is large. The central angle is any angle greater than 0 degrees and less than 360 degrees, and may further be (a) any angle greater than 0 degrees and equal to or less than 180 degrees, (b) any angle greater than 0 degrees and equal to or less than 90 degrees, (c) any angle greater than 0 degrees and equal to or less than 45 degrees, or (d) any angle equal to or greater than 0.5 degrees and less than 360 degrees, and further, (e) any angle equal to or greater than 0.5 degrees and equal to or less than 180 degrees, (f) any angle equal to or greater than 0.5 degrees and equal to or less than 90 degrees, (g) any angle equal to or greater than 0.5 degrees and equal to or less than 45 degrees, (h) any angle equal to or greater than 0.5 degrees and equal to or less than 25 degrees, or (i) any angle equal to or greater than 2 degrees and less than 360 degrees, further, (j) any angle equal to or greater than 2 degrees and equal to or less than 180 degrees, (k) any angle equal to or greater than 2 degrees and equal to or less than 90 degrees, (l) any angle equal to or greater than 2 degrees and equal to or less than 45 degrees, (m) any angle equal to or greater than 2 degrees and equal to or less than 25 degrees, or (n) any angle equal to or greater than 5 degrees and less than 360 degrees, further, (o) any angle equal to or greater than 5 degrees and equal to or less than 180 degrees, (p) any angle equal to or greater than 5 degrees and equal to or less than 90 degrees, (q) any angle equal to or greater than 5 degrees and equal to or less than 45 degrees, or (r) any angle equal to or greater than 5 degrees and equal to or less than 25 degrees. Furthermore, when it is considered about attachment and detachment of an application target, to which the vector potential is applied, to and from the VP coil 11 from the inner side of the curvature, it is preferred that the opening 14 is to be large (in other words, a curvature radius of the above-mentioned coil axis and/or the above-mentioned central angle are determined according to the shape and size of the application target).

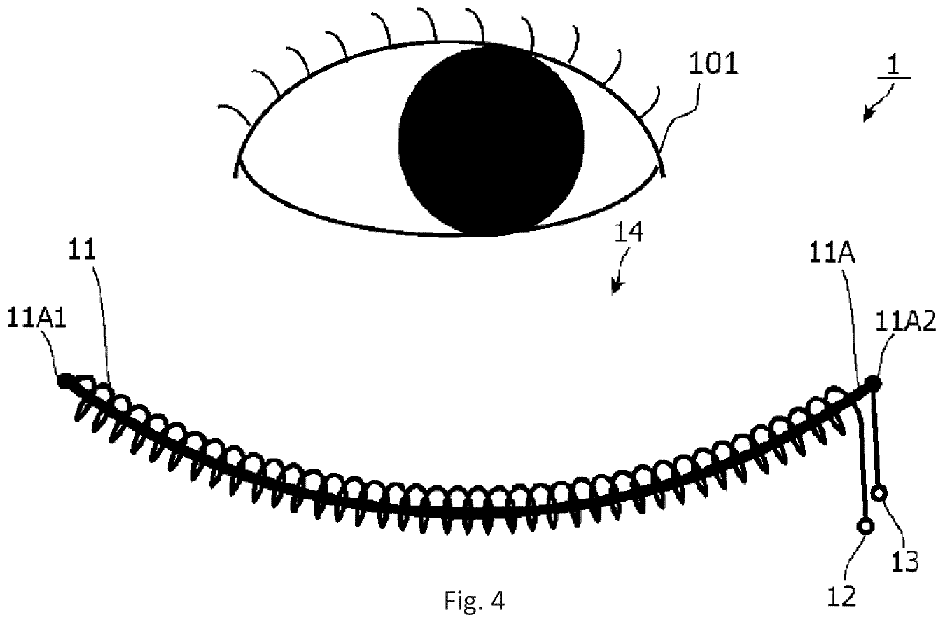

FIG. 4 is a diagram that shows an example of an application of a vector potential by a vector potential generation device 10 according to the first embodiment. As shown in, for instance, FIG. 4, the application target (in FIG. 4, an eye 101 of a human body), to which the vector potential is applied, is arranged at an outside of the opening 14.

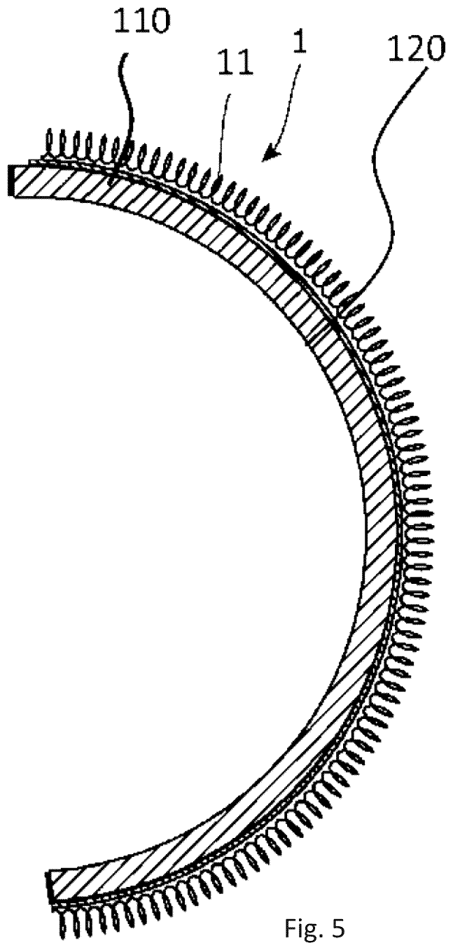

FIG. 5 is a diagram that shows an example of a support body 110 of the vector potential coil device 1 according to the first embodiment.

The vector potential coil device 1 shown in FIG. 5 has the support body 110, the vector potential coil 11, a power supply device 2 (not shown), and a control part 3 (not shown).

The support body 110 is a member having an open recessed space capable of accommodating a part of a living body and has an irradiation surface 120 facing the living body. In the first embodiment, the support body 110 is a pipe-shaped member. However, the support body 110 may also be, for instance, a plate-shaped flat member, an arc-shaped member, or a hemispherical member. Further, the above-mentioned living body may be a human or an animal.

The support body 110 may be partially made of a light-transmitting material (member) (for instance, a transparent resin material (member)). In this way, a further (additional) phototherapy element can be mounted on the vector potential coil device 1, and electrical and optical stimulations can be applied to the living body at the same time.

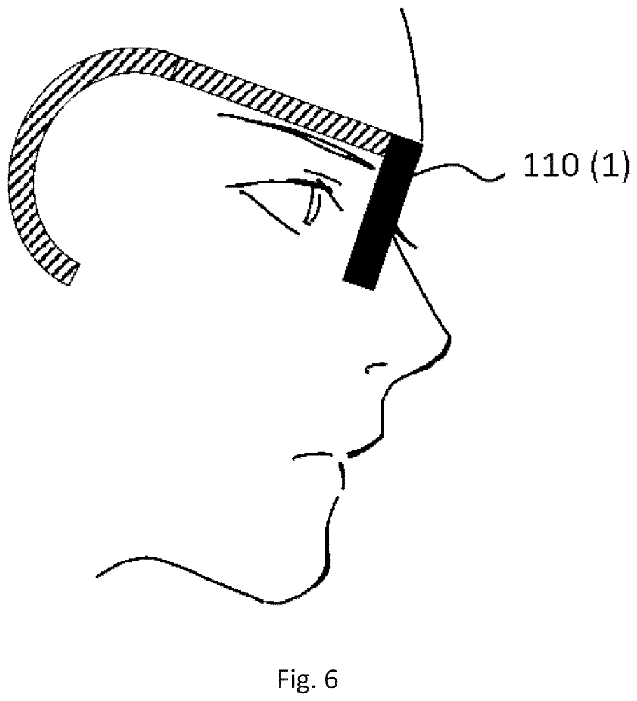

FIG. 6 is a diagram that shows an example of how to attach the vector potential coil device 1 according to the first embodiment. Without attaching the support body 110 to any part of a living body (the human body in FIG. 6), the vector potential coil 11 and the ferromagnetic member 11A are placed at a position where a vector potential is generated in the living body.

The vector potential coil device 1 shown in FIG. 6 has the form of eyeglasses, in which it is supported by the support body 110 and the support body 110 is attached to the ears by attachment parts. As a result, the vector potential coil device 1 can be carried around without taking up much space. In particular, for the purpose of the miniaturization, it is preferred to integrate the power supply device 2 (not shown) and the control part 3 (not shown) entirely or in part with the support body 110 or the attachment parts.

In addition, in the first embodiment, the ferromagnetic member 11A is formed with a conductive material such as permalloy. Further, since one end of the VP coil and one end (an end 11A1) of the ferromagnetic member 11A are electrically connected to each other, the ferromagnetic member 11A forms a path for a current. In addition, the power supply device 2 conducts a current through the VP coil 11 by applying a voltage to the other end of the VP coil 11 and the other end of the ferromagnetic member 11A. Here, the power supply device 2 conducts the current through the VP coil 11 applying the voltage to a terminal being electrically connected to the other end of the VP coil 11 and a terminal 13 being electrically connected to the other end (an end 11A2) of the ferromagnetic member 11A.

In addition, since the coil axis of the VP coil 11 does not go around once (one revolution) or more, the distance between both ends of the VP coil 11 is large. However, since the ferromagnetic member 11A is used as the current path and two of the terminals 12 and 13 are arranged on either end side of the VP coil 11, the area being encircled by the path through the wiring from the power supply device 2 to the VP coil 11 and the ferromagnetic member 11A is relatively narrow. As a result, an unnecessary magnetic field being generated due to the current flowing through the e wiring can be suppressed.

Next, an operation of the vector potential generation device 10 according to the first embodiment will be explained.

The power supply device 2, under the control of the control part 3 (not shown), alternately applies predetermined positive and negative pulse voltages to the above-mentioned terminals 12 and 13 of the vector potential generation device 10 so as to conduct a current through the VP coil 11 and the ferromagnetic member 11A.

A magnetic field is generated along the coil axis by the current being conducted through the VP coil 11. A vector potential is generated in parallel to the current. Further, an intensity of the vector potential in the inner side of the curvature of the VP coil 11 (i.e., in a periphery of the opening 14) becomes greater than the vector potential in an outer side of the curvature of the VP coil 11.

Therefore, the vector potential is effectively applied to the application target of the vector potential that is arranged in the opening 14. Further, with respect to the living body that is the application target, to which the vector potential is applied, cells, for instance, wound skeletal muscles around an eye (e.g., the orbicularis oculi muscle), a cornea, a retina, an eyelid, a vitreous body, a lacrimal sac, and a nasolacrimal duct, receive the electrical stimulation. Since an SOS signal from the damaged cells is carried by the current of this electrical stimulation, it is easier for the brain to detect it. At the same time, the brain issues a command to the body to produce more ATP (an energy source) needed for the cell repair through the self-healing mechanism of the living body.

As mentioned above, according to the above-mentioned first embodiment, the VP coil 11 is the solenoid coil extending along the curved coil axis. The ferromagnetic member 11A extends along the coil axis within the solenoid coil. The power supply device 2 conducts the current through the VP coil 11. In addition, the VP coil 11 and the ferromagnetic member 11A have the opening 14 in the circumferential direction (that is, in the circumferential direction of the VP coil 11 and the ferromagnetic member 11A that go around less than once (one revolution)).

As a result, since it is possible to arrange the application target of the vector potential at the opening 14 or in an internal or external region of the VP coil 11 via the opening 14, there are fewer restrictions on the application target of the vector potential. Further, it can also be said that the smaller the central angle is, the fewer restrictions there are on the application target of the vector potential.

For instance, by using the VP coil 11 as shown in the first embodiment, it is possible to effectively apply a vector potential to a specific location with a relatively small VP coil 11.

### Second Embodiment

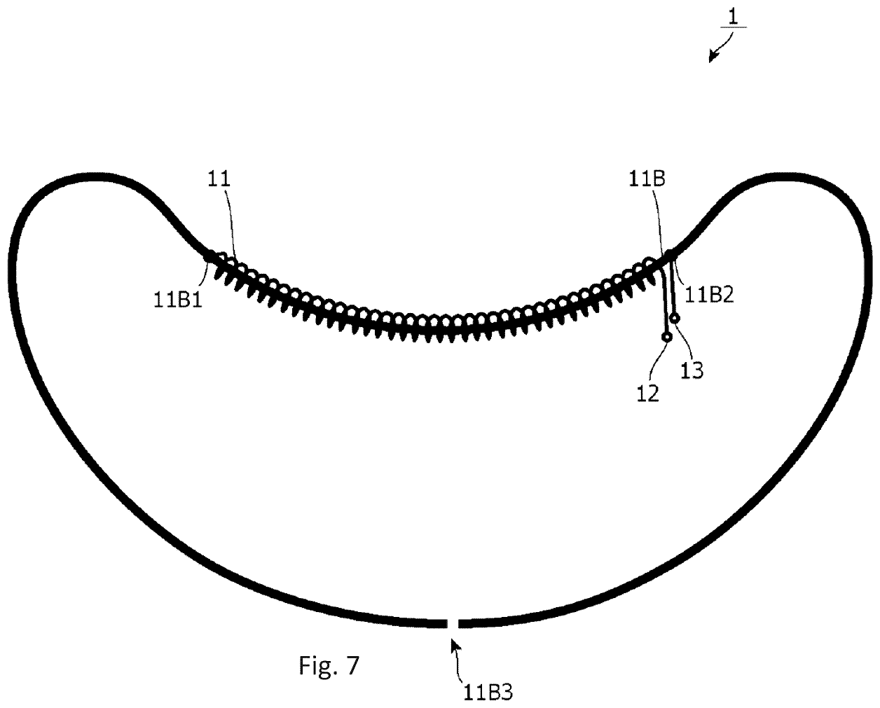

FIG. 7 is a diagram that shows a configuration of a vector potential coil device 1 with respect to a vector potential generation device 10 according to a second embodiment of the present invention.

In the second embodiment, as shown in, for instance, FIG. 7, a ferromagnetic member 11B is arranged at an inside of a VP coil 11 (i.e., a solenoid coil). In the same manner as the ferromagnetic member 11A, the ferromagnetic member 11B is in a shape along the coil axis of the VP coil 11. In addition, the ferromagnetic member 11B extends toward an outside of the VP coil 11 (in the outer side (direction) of the curvature) so as to form a closed magnetic path.

The ferromagnetic member 11B is a member being made of a conductive ferromagnetic material (for instance, a metallic magnetic material such as permalloy), and has a connection point 11B1 on a side of one coil end of the VP coil 11 and a connection point 11B2 on a side of the other coil end of the VP coil 11. Further, the one coil end of the VP coil 11 is electrically connected to the ferromagnetic member 11B at the connection point 11B1.

In addition, the other coil end of the VP coil 11 is electrically connected to a terminal 12. The connection point 11B2 of the ferromagnetic member 11B is electrically connected to a terminal 13 via a lead wire. Further, the power supply device 2 applies a voltage to the terminals 12 and 13 so as to conduct a current through the VP coil 11.

In addition, a gap 11B3 is formed in the ferromagnetic member 11B at the outer side of the curvature of the VP coil 11. Further, the gap 11B3 prevents the current from being conducted through an outside part of the curvature of the VP coil 11 in the ferromagnetic member 11B.

Further, it is preferred that the transition portion between the inner portion and the outer portion of the ferromagnetic member 11B is made into a continuous and smooth curve shape without any steep bend portions, in order to reduce a leakage of a magnetic flux and reduce the influence of the decrease of the magnetic permeability due to bending processing. Moreover, the ferromagnetic member 11B may be formed by connecting a plurality of members.

The vector potential coil device 1 with respect to the vector potential generation device 10 according to the second embodiment can be applicable to the first embodiment.

### Third Embodiment

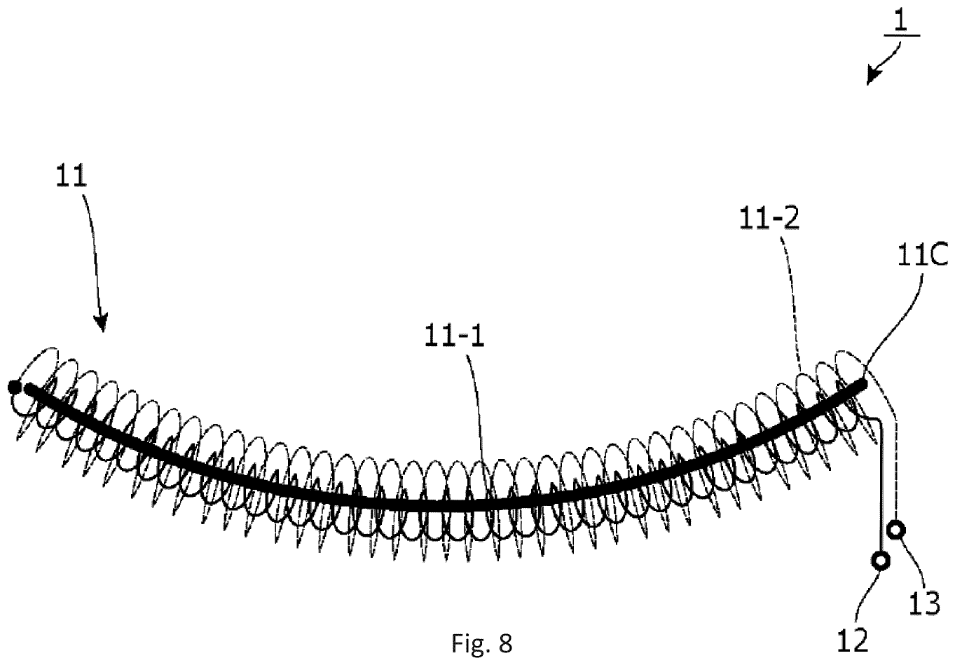

FIG. 8 is a diagram that shows a configuration of a vector potential coil device 1 with respect to a vector potential generation device 10 according to a third embodiment of the present invention.

In the third embodiment, as shown in, for instance, FIG. 8, the VP coil 11 has an inner solenoid coil 11-1 and an outer solenoid coil 11-2 that respectively extend along the same curved coil axis and in which coil diameters are different from each other. Further, one coil end of the inner solenoid coil 11-1 and one coil end of the outer solenoid coil 11-2 are electrically connected. Each of the inner solenoid coil 11-1 and the outer solenoid coil 11-2 functions as one VP coil. Therefore, the VP coil 11 according to the third embodiment electrically has a configuration in which two VP coils are connected in series and in the same phase.

Furthermore, in the third embodiment, as shown in, for instance, FIG. 8, a ferromagnetic member 11C is arranged at an inside of the VP coil 11 (the inner solenoid coil 11-1). The ferromagnetic member 11C is the same as the above-mentioned ferromagnetic member 11A. However, the VP coil 11 and the ferromagnetic member 11C are not electrically connected. Further, the ferromagnetic member 11C may not have to be conductive.

The power supply device 2 applies a voltage to the other end of the inner solenoid coil 11-1 and the other end of the outer solenoid coil 11-2 so that the current is conducted through the VP coil 11. Specifically, the power supply device 2 applies the voltage to the terminal 12 being electrically connected to the other end of the inner solenoid coil 11-1 and the terminal 13 being electrically connected to the other end of the outer solenoid coil 11-2 so that the current is conducted through the VP coil 11.

The vector potential coil device 1 with respect to the vector potential generation device 10 according to the third embodiment can be applicable to any of the first to second embodiments.

### Fourth Embodiment

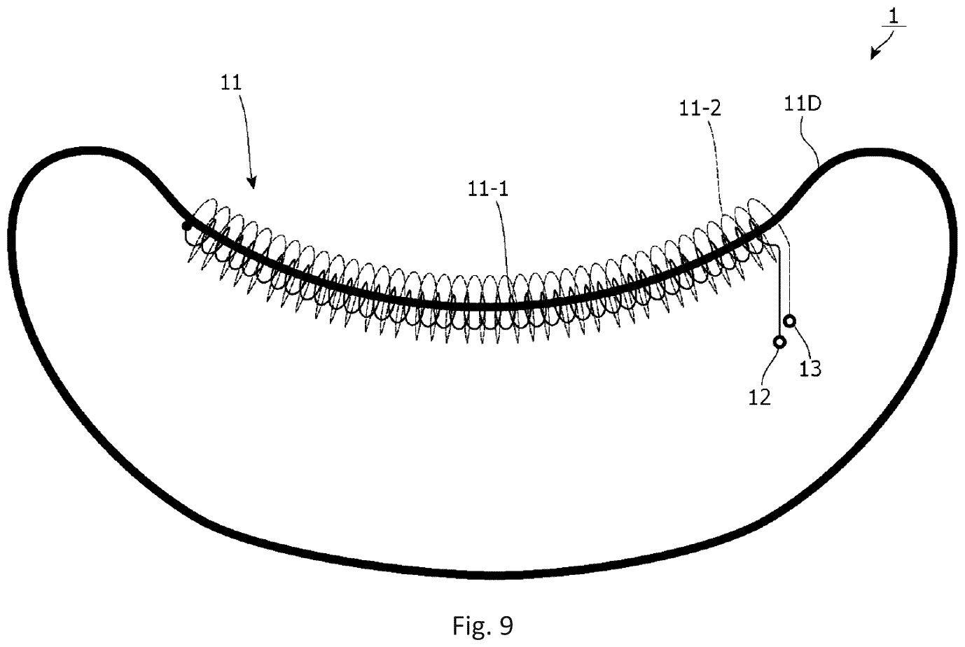

FIG. 9 is a diagram that shows a configuration of a vector potential coil device 1 with respect to a vector potential generation device 10 according to a fourth embodiment of the present invention.

In the fourth embodiment, as shown in, for instance, FIG. 9, the VP coil 11 has the inner solenoid coil 11-1 and the outer solenoid coil 11-2 that are the same as those in the third embodiment. Furthermore, in the fourth embodiment, as shown in, for instance, FIG. 9, a ferromagnetic member 11D is arranged at an inside of the VP coil 11 (the inner solenoid coil 11-1). The ferromagnetic member 11D is the same as the above-mentioned ferromagnetic member 11B. However, the VP coil 11 and the ferromagnetic member 11D are not electrically connected. Further, the ferromagnetic member 11D may not have to be conductive. In addition, a gap is not provided. That is, in the fourth embodiment, since an AC current is conducted through the inner solenoid coil 11-1 and the outer solenoid coil 11-2 but is not conducted through the ferromagnetic member 11D, the ferromagnetic member 11D does not require conductivity or the gap.

Note the that other configurations and operations of the vector potential coil device 1 with respect to the vector potential generation device 10 according to the fourth embodiment are the same as those explained in the third embodiment. Therefore, the explanations of them will be omitted.

### Fifth Embodiment

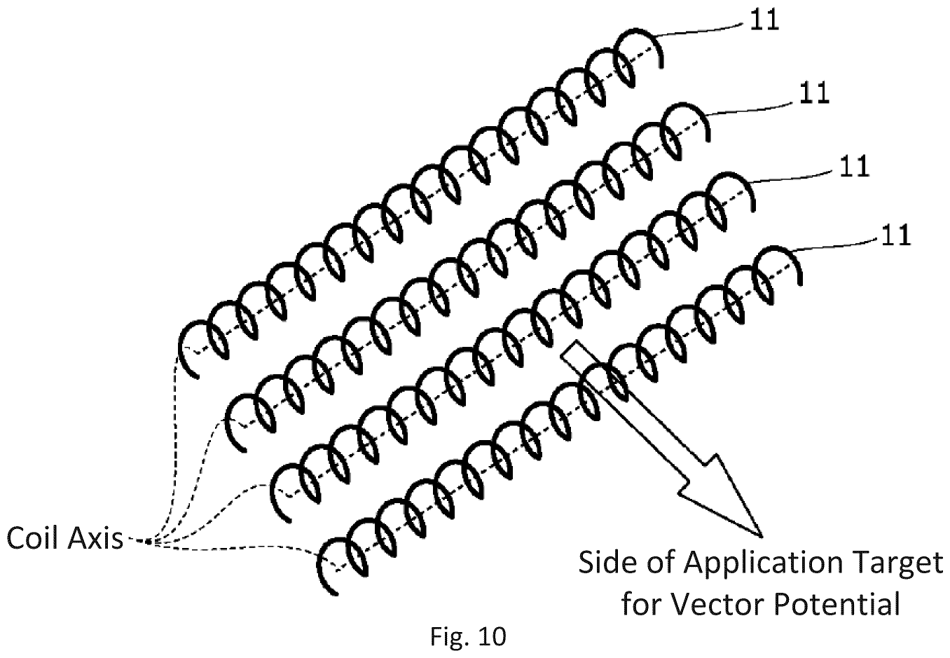

FIG. 10 is a diagram that shows a configuration of a vector potential coil device 1 with respect to a vector potential generation device 10 according to a fifth embodiment of the present invention.

In the fifth embodiment, the vector potential coil device 1 has a plurality of VP coils 11. Each VP coil 11 in the fifth embodiment has a linear (straight) coil axis. Further, the VP coils 11 are a plurality of solenoid coils extending along the coil axes. The plurality of VP coils 11 are arranged along a linear arrangement direction. In other words, the outer shape of the vector potential coil device 1 is in a substantially flat plate shape. The power supply device 2 conducts a current through the plurality of VP coils 11. Further, the plurality of VP coils 11 may be electrically connected in series or in parallel to one another. Furthermore, a plurality of power supply devices 2 may conduct the current to the plurality of VP coils 11, respectively. In this case, the plurality of power supply devices 2 respectively conduct an AC current through the plurality of VP coils 11 under the condition in which the AC currents being conducted through the plurality of VP coils 11 are synchronized. Furthermore, the vector potential coil device 1 has a plurality of ferromagnetic members (not shown) that extend along the coil axes of the plurality of VP coils 11 in the same manner as the above-mentioned ferromagnetic members.

As mentioned above, by providing the plurality of VP coils 11, the intensity of the vector potential being applied to the application target becomes greater.

Note the that other configurations and operations of the vector potential generation device 10 according to the fifth embodiment are the same as those explained in any of the other embodiments. Therefore, the explanations of them will be omitted.

### Sixth Embodiment

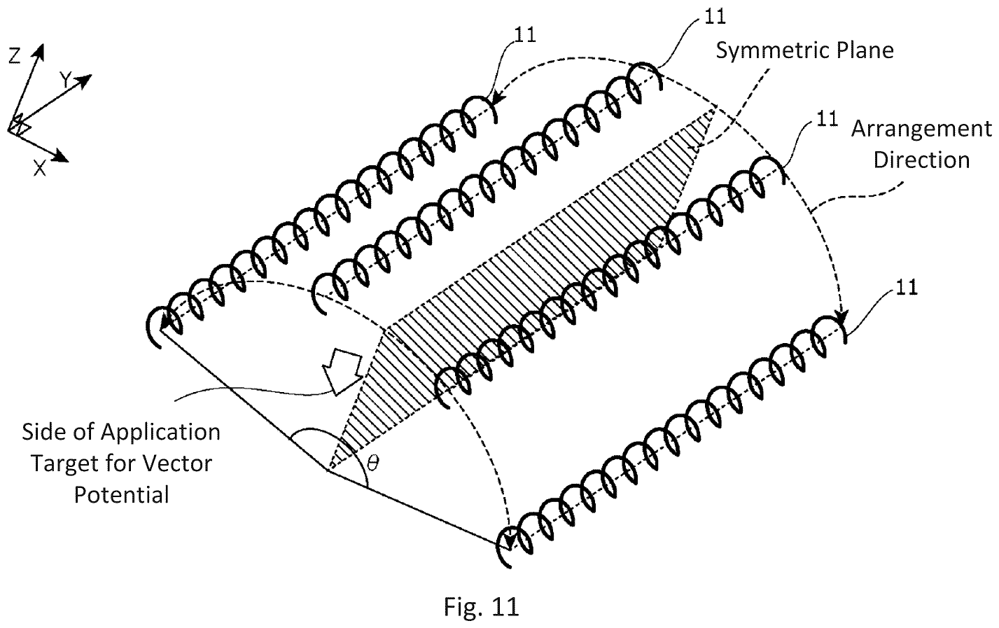

FIG. 11 is a diagram that shows a configuration of a vector potential coil device 1 with respect to a vector potential generation device 10 according to a sixth embodiment of the present invention.

In the sixth embodiment, the vector potential coil device 1 has a plurality of VP coils 11. Each VP coil 11 in the sixth embodiment has a linear coil axis. Further, the VP coils 11 are a plurality of solenoid coils extending along the coil axes. The plurality of VP coils 11 are arranged along a curved (curvature) arrangement direction. The power supply device 2 conducts the current through the plurality of VP coils 11. Further, the plurality of VP coils 11 may be electrically connected in series or in parallel to one another. Furthermore, the vector potential coil device 1 has a plurality of ferromagnetic members (not shown) that extend along the coil axes of the plurality of VP coils 11 in the same manner as the above-mentioned ferromagnetic members. Here, the arrangement direction is a closed curve. Thus, the plurality of VP coils 11 are arranged along the circular-arc-shaped arrangement direction. In particular, the plurality of VP coils 11 are arranged within a range of a predetermined central angle θ (here, at an interval of an equal angle) with respect to a circle that includes the circular arc in the arrangement direction. Since the vector potentials of two of the VP coils 11 are canceled out at the intermediate position between the two VP coils 11, for instance, the central angle θ is set to be any angle less than 180 degrees.

For instance, a part of the human body, such as all or a part of an eye, the muscles around the eye, or acupressure points related to the eye, is arranged within or at an outside of the space in the inner side (direction) of the arranged plurality of VP coils 11. Thus, the vector potential is applied to that part.

Note that as shown in, for instance, FIG. 11, when the plurality of VP coils 11 having the linear coil axes are arranged plane-symmetrically with respect to a predetermined symmetric plane (a plane perpendicular to the X-axis and parallel to the Z-axis and the Y-axis) along the curved arrangement direction, on an axis that passes through the center of the circle that includes the circular arc in the arrangement direction, and at the same time, is parallel to the coil axes, as a result of a vector synthesis of the vector potentials that are generated by the plurality of VP coils 11, the vector potential is generated in a vertical (perpendicular) direction with respect to the symmetric plane (the X-axis direction in FIG. 11). Therefore, for instance, by combining the VP coil 11 having the curved coil axis shown in FIG. 4 with the plurality of VP coils 11 having the linear coil axes and being arranged plane-symmetrically with respect to the predetermined symmetric plane along the curved arrangement direction, it is possible to generate a vector potential in a desired direction within a two-dimensional plane of the X-axis and Y-axis.

Note that the other configurations and operations of the vector potential generation device 10 according to the sixth embodiment are the same as those explained in any of the other embodiments. Therefore, the explanations of them will be omitted.

### Seventh Embodiment

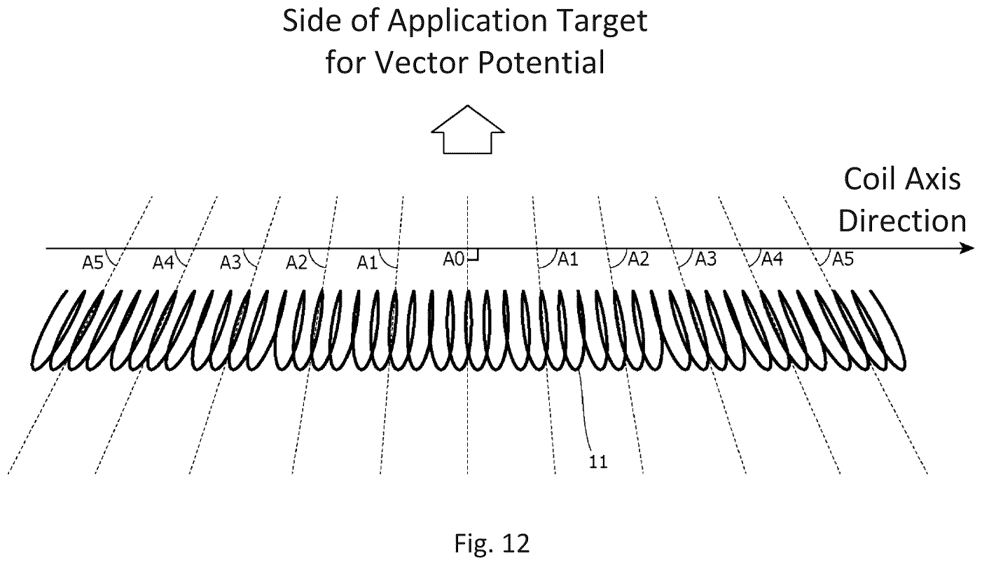

FIG. 12 is a diagram that shows a configuration of a vector potential coil device 1 with respect to a vector potential generation device 10 according to a seventh embodiment of the present invention.

In the seventh embodiment, the vector potential coil device 1 has a plurality of VP coils 11. Each of the plurality of VP coils 11 is wound along (around) a linear coil axis and is wound so that inclination angles in the winding direction gradually change along the direction of the coil axis. Further, the vector potential coil device 1 has a plurality of ferromagnetic members (not shown) that extend along the coil axes of the plurality of VP coils 11 in the same manner as the above-mentioned ferromagnetic members.

In the seventh embodiment, as shown in, for instance, FIG. 12, the VP coil 11 is wound along (around) the linear coil axis, and is wound so that the inclination angles (the angles formed by the coil axis direction and the winding direction) A0-A5 in the winding direction gradually change along the direction of the coil axis. Specifically, the inclination angle at the center of the VP coil 11 is 90 degrees. Further, the inclination angle becomes smaller as it moves away from the center (A0>A1>A2>A3>A4>A5). As a result, the above-mentioned vector potential can be applied with satisfactory intensity in the same manner as the case of the curved VP coil 11.

Note other configurations and that the operations of the vector potential generation device 10 according to the seventh embodiment are the same as those explained in any of the other embodiments. Therefore, the explanations of them will be omitted.

### Eighth Embodiment

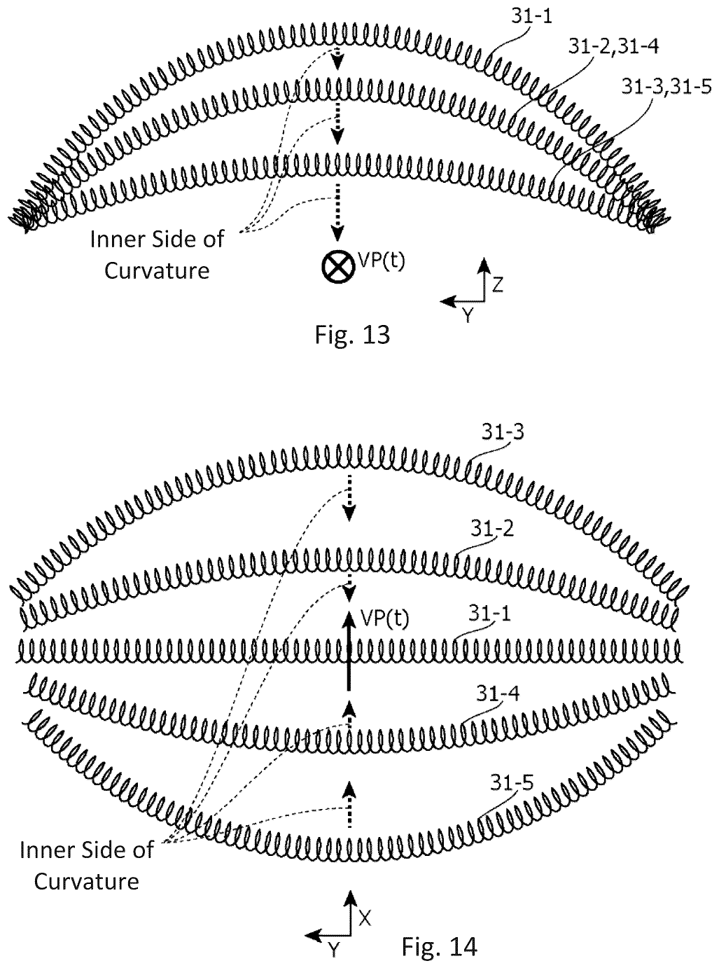

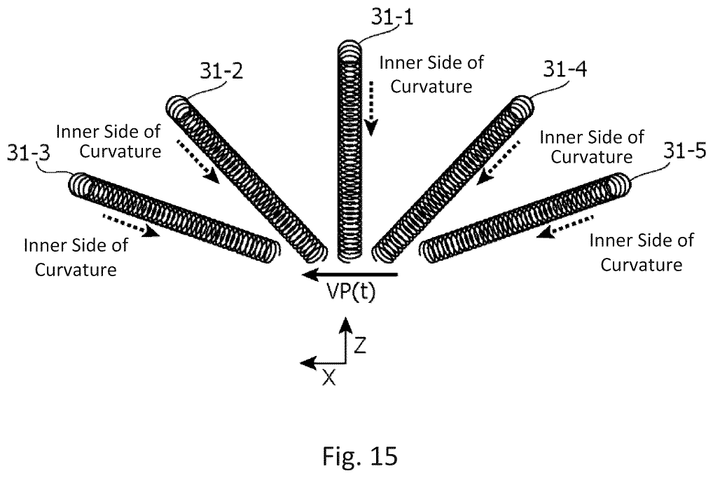

FIG. 13 is a front view that shows an example of a vector potential coil with respect to a vector potential coil device 1 according to an eighth embodiment of the present invention. FIG. 14 is a top view that shows an example of the vector potential coil with respect to the vector potential coil device 1 according to the eighth embodiment of the present invention. FIG. 15 is a side view that shows an example of the vector potential coil with respect to the vector potential coil device 1 according to the eighth embodiment of the present invention.

The vector potential coil device 1 according to the eighth embodiment has a plurality of vector potential coils 31-1-31-5. As shown in, for instance, FIGS. 13-15, the plurality of vector potential coils 31-1-31-5 are respectively wound along (around) a curved coil axis, and are arranged so that the inner sides (directions) of the curvatures of the coil axes (in other words, planes that include the coil axes) cross mutually. For instance, as shown in FIG. 15, the plurality of vector potential coils 31-1-31-5 are arranged so that the planes that include the coil axes of the plurality of vector potential coils 31-1 to 31-5 are parallel to the Y-axis direction, and at the same time, the angle intervals of the inclination angles of these planes with respect to the X-axis direction are substantially the same. In addition, here, the inclination angle of the vector potential coil 31-1 is 90 degrees.

Note that, here, the vector potential coil device 1 has five of the vector potential coils 31-1-31-5. However, the vector potential coil device 1 may have the vector potential coils 31-1-31-M in the same manner as the configuration described above. The number M is either 2-4 coils or 6 or more coils.

For instance, the shape (such as the curvature) and the arrangement of the coil axes are determined so that the coil axes of the plurality of vector potential coils 31-1-31-5 are included in a single partial spherical surface (for instance, a semispherical surface). Further, the application target is arranged at the center of the spherical surface that includes that partial spherical surface (in other words, the center of curvatures of all of the coil axes). Further, the shape (such as the curvature) and the arrangement of the coil axes may be determined so that the coil axes of the plurality of vector potential coils 31-1-31-5 are included in a curved surface (a partial aspherical surface) other than a single partial spherical surface.

Further, the plurality of vector potential coils 31-1-31-5 respectively generate a vector potential according to the AC current in the same manner as the above-mentioned embodiments. The vector potentials by the plurality of vector potential coils 31-1-31-5 are synthesized so that a vector potential VP (t) is obtained. Here, the power supply device 2 conducts the AC current through the plurality of vector potential coils 31-1-31-5 so that the amplitude of the synthesized vector potential VP (t) becomes maximum (for instance, in the same phase mutually).

Note that the other configurations and operations of the vector potential generation device 10 according to the eighth embodiment are the same as those explained in any of the other embodiments. Therefore, the explanations of them will be omitted. In other words, any of the above-mentioned ferromagnetic members may be respectively arranged along the coil axes of the plurality of vector potential coils 31-1-31-5.

As mentioned above, according to the vector potential generation device 10 according to the above-mentioned eighth embodiment, it is possible to concentrate the vector potentials in the inner sides (direction) of the curvatures of the plurality of vector potential coils 31-1-31-5 and apply a high-intensity vector potential to the application target.

### Ninth Embodiment

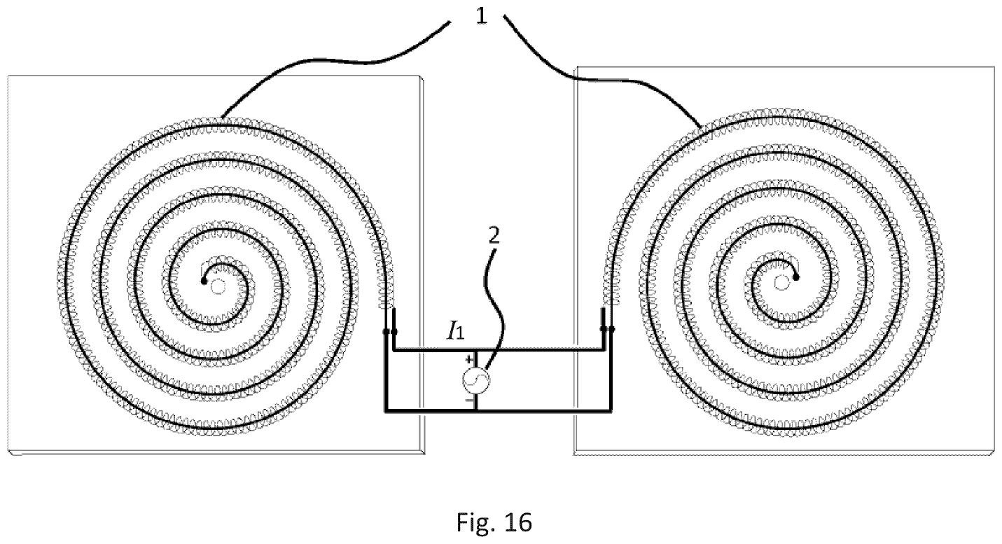

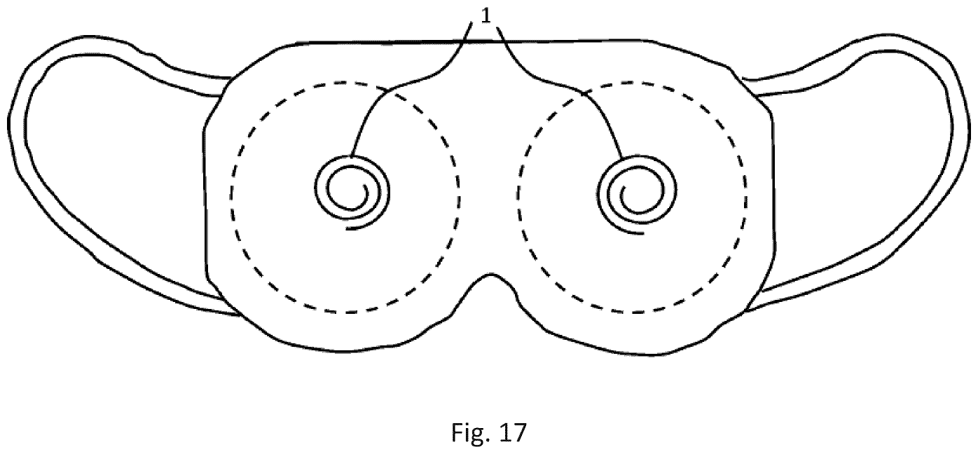

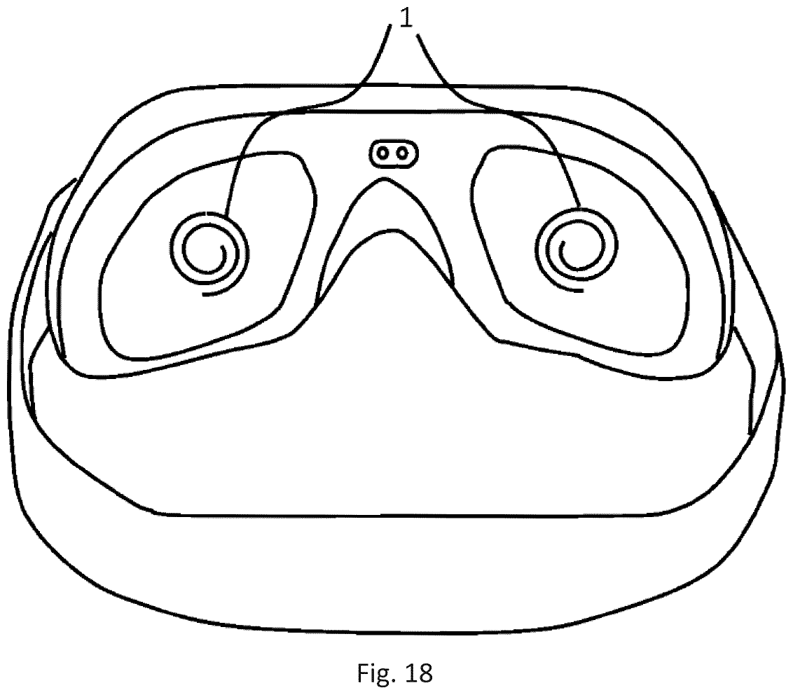

FIG. 16 is a diagram that shows an example of a vector potential coil with respect to a vector potential coil device 1 according to a ninth embodiment of the present invention. FIG. 17 is a diagram that shows an example of attaching the vector potential coil device 1 according to the ninth embodiment of the present invention. FIG. 18 is a diagram that shows another example of attaching the vector potential coil device 1 according to the ninth embodiment of the present invention.

The vector potential coil device 1 according to the ninth embodiment has two vector potential coils. As shown in, for instance, FIG. 16, the two vector potential coils are respectively wound along a spiral coil axis in two or more layers. Further, the two vector potential coils are arranged in parallel (side by side). Here, two or more layers refer to a shape obtained by making the vector potential coil go around once on the same plane to form a circular shape and perform it two or more times. Further, the spiral shape differs from a concentric shape and is a curved shape that moves away from the center as it turns, like a whirlpool (vortex). There are two types of swiveling (turning or rotating) directions which are clockwise and counterclockwise directions. Furthermore, when the number of layers of the vector potential coil according to the ninth embodiment may be even one and the winding directions of the two vector potential coils are opposite, the effects of the present invention can be obtained.

Note that here, the vector potential coil device 1 has two vector potential coils, and the swiveling (turning or rotating) directions of the vector potential coils are exactly opposite to each other. In other words, the swiveling direction of one of the vector potential coils is the clockwise direction and the other of the vector potential coils is the counterclockwise direction. As a result, a vector potential can be generated at a predetermined position in the living body. For instance, when an AC current flows, an electrical stimulation is applied to one eyeball in a direction away from the surface of the paper (from the surface of the paper toward the back of the surface of the paper), and the electrical stimulation is applied to the other eyeball in the opposite direction, from the back of the surface of the paper toward the surface of the paper. In addition, when the direction of the AC current is changed, the electrical stimulation is applied to one eyeball in the direction from the back of the surface of the paper toward the surface of the paper, and the electrical stimulation is applied to the other eyeball in the opposite direction, from the surface of the paper toward the back of the surface of the paper. In this way, the two vector potentials are always cancelled out at a position far away with a distance, and the two vector potentials can be applied alternately to positions of near fields of the two vector potential coils. For instance, in the ophthalmic treatments, the stimulation of the vector potential is applied to the eyeball that exists nearby. Further, the stimulation is cancelled out to the brain that exists in the distance by the differential stimulation so as to reduce or suppress the side effects.

In this way, even in the situation in which an optical approach to the retina (an observation with a camera, and a laser treatment) is difficult due to the clouding of the lens or the vitreous body of the eye, or the light scattering, when using the vector potential coil device 1 according to the ninth embodiment of the present invention, it is possible to apply the electrical stimulation evenly without being affected by the medium (target). In particular, in the vitreous body region after the surgery of the retinal detachment, during the process in which the xenon hexafluoride (XeF6) gas is replaced by the body fluids, a liquid surface exists within the eye. Thus, it is difficult to perform the optical approach with respect to the retina. Further, in the electrostatic induction method, it is extremely difficult for an electric field to reach the retina through the highly conductive vitreous body. On the other hand, when the vector potential coil device 1 according to the ninth embodiment of the present invention is utilized, it is possible to generate the vector potential in the retina such that it is possible to apply the electrical stimulation as expected.

In addition, FIG. 17 is the diagram that shows the example of attaching the vector potential coil device 1 according to the ninth embodiment of the present invention. In this example, the vector potential coil device 1 according to the ninth embodiment of the present invention is attached to a frame body such as an eye mask. As a result, since the vector potential coil device 1 is arranged in the vicinity of the eyeball as mentioned above, it is possible to generate the vector potential in the part (site) such as the retina.

FIG. 18 is the diagram that shows another example of attaching the vector potential coil device 1 according to the ninth embodiment of the present invention. In this example, the vector potential coil device 1 according to the ninth embodiment of the present invention is attached to a frame body such as goggles. Since the vector potential coil device 1 is arranged in the vicinity of the eyeball in the same manner as the case mentioned above, it is possible to generate the vector potential in the part (site) such as the retina.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

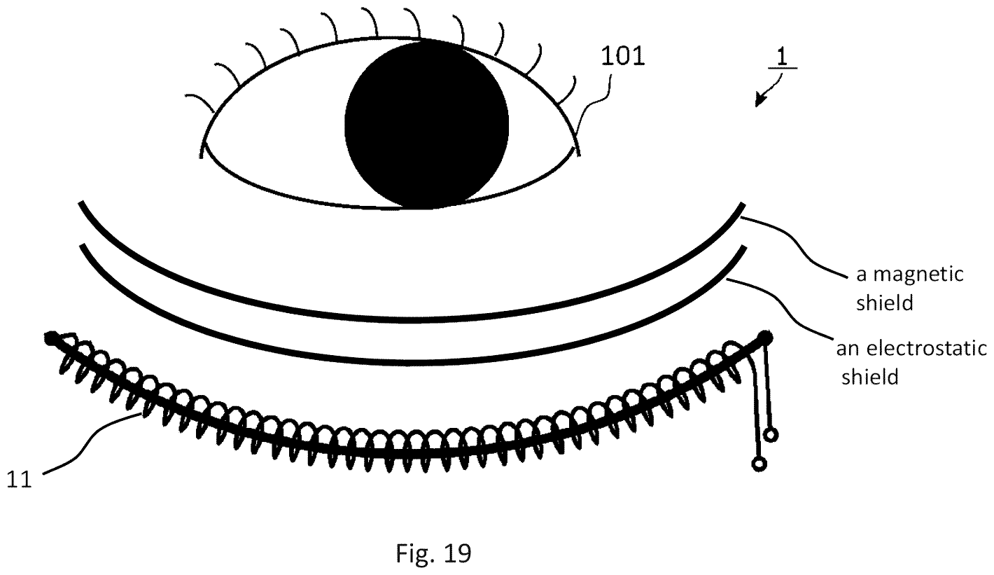

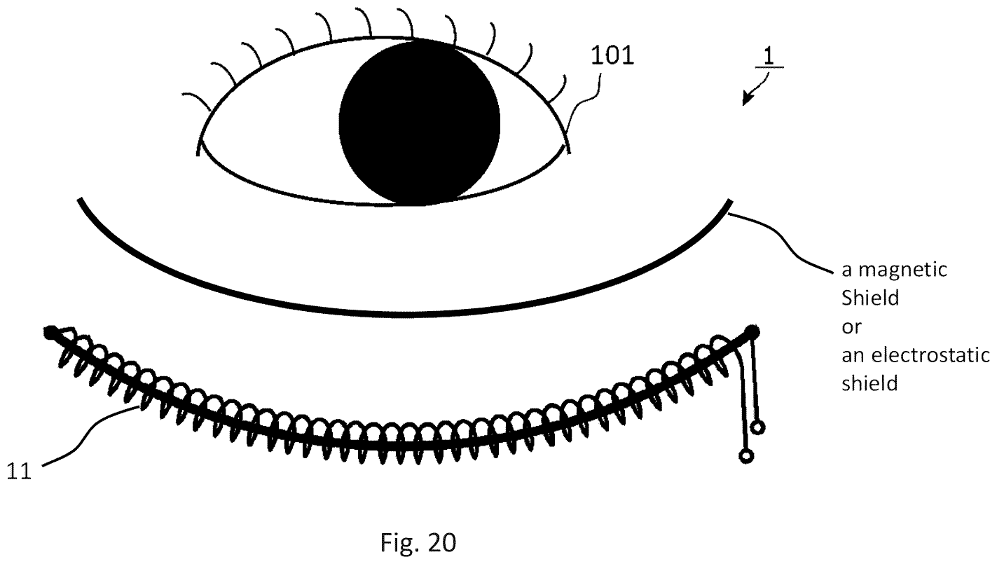

For instance, in the above-mentioned embodiments, there may be one of or both an electrostatic shield and a magnetic shield between the VP coil 11 and the application target of the vector potential (for instance, the eye 101). Specifically, as shown in, for instance, FIG. 19, the vector potential coil device 1 has the VP coil, the electrostatic shield (between the VP coil and the eye), and the magnetic shield (between the electrostatic shield and the eye). Note, however, that the positions of the magnetic shield and the electrostatic shield may be switched between the VP coil 11 and the eye. Further, as shown in, for instance, FIG. 20, the vector potential coil device 1 has the VP coil and either the electrostatic shield or the magnetic shield (between the VP coil 11 and the eye). In addition, the other features shown in the other embodiments can be combined with or added to the configurations shown in FIGS. 19 and 20. Even in this case, the vector potential penetrates one of or both the electrostatic shield and the magnetic shield. Thus, even when one of or both the electrostatic shield and the magnetic shield exists, the vector potential can be applied to the application target of the vector potential.

Further, in the above-mentioned embodiments, the ferromagnetic member arranged along the coil axis of the VP coil 11 may be omitted as necessary.

In addition, in the above-mentioned third and fourth embodiments, the VP coil 11 has the two-layer structure, which are the inner solenoid coil 11-1 and the outer solenoid coil 11-2, in the radial direction. However, the number of layers may be four or more as long as the number of layers is an even number. In this case, either one of the ends of the solenoid coil 11-i is connected to the solenoid coil 11-(i+1) in the next layer so that the solenoid coils 11-i in all layers are electrically connected in series.

### INDUSTRIAL APPLICABILITY

The present invention can be applicable to, for instance, the generation of a vector potential by using a VP coil in an electrical stimulation method for use in ophthalmic treatments.

What is claimed is:

1. An electrical stimulation vector potential generation device for use in an ophthalmic treatment, comprising:
a vector potential coil which is a solenoid coil extending along a coil axis that is straight or curved;
a ferromagnetic member which extends along the coil axis in an inside of the solenoid coil; and
a power supply device which conducts a current through the vector potential coil,
wherein the vector potential coil and the ferromagnetic member have openings in a circumferential direction.

2. The electrical stimulation vector potential generation device for use in the ophthalmic treatment according to claim 1, further comprising:
a plurality of vector potential coils, which are solenoid coils respectively extending along a coil axis that is straight or curved, including the vector potential coil; and
a plurality of ferromagnetic members which extend along the coil axes in insides of the solenoid coils of the plurality of vector potential coils, respectively,
wherein the power supply device conducts the current through the plurality of vector potential coils.

3. The electrical stimulation vector potential generation device for use in the ophthalmic treatment according to claim 1,
wherein the coil axis is arc-shaped, and
an angle between one end and the other end of the vector potential coil, when viewed from a center of a circle that includes the coil axis, is less than 360 degrees.

4. The electrical stimulation vector potential generation device for use in the ophthalmic treatment according to claim 1,
wherein the ferromagnetic member is electrically conductive,
one end of the vector potential coil and one end of the ferromagnetic member are electrically connected to each other, and
the power supply device applies a voltage to the other end of the vector potential coil and the other end of the ferromagnetic member to conduct the current through the vector potential coil.

5. The electrical stimulation vector potential generation device for use in the ophthalmic treatment according to claim 1,
wherein the ferromagnetic member extends to an outside of the vector potential coil to form a closed magnetic path.

6. The electrical stimulation vector potential generation device for use in the ophthalmic treatment according to claim 1,
wherein the vector potential coil is configured with an inner solenoid coil and an outer solenoid coil each extending along the coil axis that is the same and curved,
one end of the inner solenoid coil and one end of the outer solenoid coil are electrically connected to each other, and
the power supply device applies a voltage to the other end of the inner solenoid coil and the other end of the outer solenoid coil to conduct the current through the vector potential coil.

7. The electrical stimulation vector potential generation device for use in the ophthalmic treatment according to claim 1, further comprising:
a support body that supports the vector potential coil and the ferromagnetic member,
wherein the support body places the vector potential coil and the ferromagnetic member at a position where a vector potential is generated in a living body, and
an electrostatic shield and a magnetic shield, or one of the electrostatic shield and the magnetic shield are positioned between the living body and the vector potential coil and the ferromagnetic member.

8. The electrical stimulation vector potential generation device for use in the ophthalmic treatment according to claim 1,
wherein the power supply device applies positive and negative pulse voltages alternately.

9. A vector potential coil arrangement method comprising:
arranging a vector potential coil and a ferromagnetic member at a position where a vector potential is generated in a living body without attaching a support body to the living body and by using the support body, the support body supporting the vector potential coil and the ferromagnetic member, the vector potential coil being a solenoid coil that extends along a coil axis that is straight or curved, the ferromagnetic member extending along the coil axis in an inside of the solenoid coil.

10. An electrical stimulation vector potential generation device for use in an ophthalmic treatment, comprising:
a plurality of vector potential coils which are a plurality of solenoid coils, respectively, each of the plurality of vector potential coils extending along each of coil axes; and
a power supply device which conducts a current through the plurality of vector potential coils,
wherein the plurality of vector potential coils are arranged along an arrangement direction that is straight or curved.

11. The electrical stimulation vector potential generation device for use in the ophthalmic treatment according to claim 10, further comprising:
a plurality of ferromagnetic members extending along the coil axes in insides of the plurality of solenoid coils, respectively.

12. The electrical stimulation vector potential generation device for use in the ophthalmic treatment according to claim 10,
wherein each of the plurality of vector potential coils is wound along the coil axis that is straight, and is wound so that inclination angles in a winding direction are gradually changed along a direction of the coil axis.

13. The electrical stimulation vector potential generation device for use in the ophthalmic treatment according to claim 10,
wherein the plurality of vector potential coils are arranged along an arrangement direction that is arc-shaped within a range of a predetermined central angle about a circle that includes an arc of the arrangement direction, and
the predetermined central angle is any angle less than 180 degrees.

14. The electrical stimulation vector potential generation device for use in the ophthalmic treatment according to claim 10,
wherein each of the plurality of vector potential coils is wound along the coil axis that is curved, and
the plurality of vector potential coils are arranged so that inner directions of the curvatures of the coil axes intersect one another.

15. The electrical stimulation vector potential generation device for use in the ophthalmic treatment according to claim 10, further comprising:
a support body which supports the plurality of vector potential coils,
wherein the support body places the plurality of vector potential coils at a position where a vector potential is generated in a living body without attaching the support body to the living body and by using the support body, and
an electrostatic shield and a magnetic shield, or one of the electrostatic shield and the magnetic shield are positioned between the living body and the vector potential coil and a ferromagnetic member.

16. The electrical stimulation vector potential generation device for use in the ophthalmic treatment according to claim 10,
wherein the power supply device applies positive and negative pulse voltages alternately.
